(19) United States
(12) Patent Application Publication
(10) Pub. No.: US 2011/0196278 A1
(43) Pub. Date: Aug. 11, 2011

(54) ADHESIVE FILM DRAPE FOR USE WITH NEGATIVE PRESSURE WOUND THERAPY DEVICE

(76) Inventors: Pal Svedman, Malmo (SE); Mark S. Meents, Germantown, MD (US); Edward M. Litzie, Sterling, VA (US)

(21) Appl. No.: 12/700,858

(22) Filed: Feb. 5, 2010

(51) Int. Cl. A61F 13/00

(52) U.S. Cl. 602/43

(57) ABSTRACT

A therapeutic adhesive film drape includes an outer transparent layer which has a printed grid across an outer surface thereof and an inner layer for application adjacent a wound which includes a semi-permeable transparent adhesive cover removably connected to the outer transparent layer. A method of treatment employs the therapeutic drape.

### BACKGROUND

1. Field of Invention

The invention is generally directed to a therapeutic device for the promotion of wound healing. More particularly, the present invention relates to an improved adhesive film drape for a therapeutic device of the type providing fluid irrigation and vacuum drainage of a wound.

2. Related Art

These devices are normally used in clinical settings such as hospitals or extended care facilities, but patients can often be located in non-clinical environments, where portability, ease of use, and control of therapy parameters is necessary. Such places can, for example, include the home, office or motor vehicles, and at the extreme, military battlefields and other locations where electrical power may be unreliable or unavailable.

Negative pressure wound therapy (NPWT), also known as vacuum drainage or closed-suction drainage, is known. A vacuum source is connected to a semi-occluded or occluded therapeutic member, such as a compressible wound dressing. Various porous dressings comprising gauze, felts, foams, beads and/or fibers can be used in conjunction with an occlusive semi-permeable cover and a controlled vacuum source. In addition to negative pressure, there exist pump devices configured to supply positive pressure to another therapeutic member, such as an inflatable cuff for various medical therapies.

In addition to using negative pressure wound therapy, many devices employ concomitant wound irrigation. For example, a known wound healing apparatus includes a porous dressing made of polyurethane foam placed adjacent a wound and covered by a semi-permeable and flexible plastic sheet. The dressing further includes fluid supply and fluid drainage connections in communication with the cavity formed by the cover, foam and skin. The fluid supply is connected to a fluid source that can include an aqueous topical anesthetic or antibiotic solution, isotonic saline, or other medicaments for use in providing therapy to the wound. The fluid drainage can be connected to a vacuum source where fluid can be removed from the cavity and subatmospheric pressures can be maintained inside the cavity. The wound irrigation apparatus, although able to provide efficacious therapy, is somewhat cumbersome, difficult to use without trained professional medical personnel, and generally impractical outside the clinical setting. Such a device does not address various factors concerning patients outside clinical settings.

Some devices use vacuum sealing of wound dressings consisting of polyvinyl alcohol foam cut to size and stapled to the margins of the wound. Such dressings are covered by a semi-permeable membrane while suction and fluid connections are provided by small plastic tubes which are introduced into the foam generally through the patient's skin. Such devices alternate in time between vacuum drainage and the introduction of aqueous medicaments to the wound site, but do not do both simultaneously. While the prior devices have proven to be useful in fixed therapeutic sites, such devices require improvement to render broader and friendlier use.

### SUMMARY OF THE INVENTION

It is an object to improve wound healing.

It is another object to improve devices for use in treating wounds.

It is an object to improve devices for treating wounds.

It is yet another object to provide a therapeutic device for treating wounds which enables wound healing progression tracking.

It is yet another object to provide a therapeutic device for treating wounds which has improved ease of use.

It is yet another object to provide an improved drape for a therapeutic device for treating wounds.

One embodiment of the invention is directed to an improved drape for therapeutic use. The drape is well suited for use with a therapeutic device for treating wounds of the type having a housing equipped with a fluid mover for one of raising, compressing, or transferring fluid, and a therapeutic member operably connected to the fluid mover and actuated thereby, the therapeutic member operably disposably used on a patient in a manner to deliver therapy to the patient as a function of actuation of the fluid mover. The therapeutic drape includes an outer transparent layer which has a printed grid across an outer surface thereof and an inner layer for application adjacent a wound which includes a semi-permeable transparent adhesive cover removably connected to the outer transparent layer. The semi-permeable transparent adhesive cover has a first surface connected to the outer layer and a second surface having a pressure sensitive adhesive material thereon. The therapeutic drape includes a release liner sheet removably connected to the second surface of the inner layer. In one embodiment, the drape includes a symmetrically printed grid across a surface thereof. The outer layer and an inner layer can be electrostatically bonded, for example. Opposing lateral edges of each layer of the drape can be fashioned in a non-stick manner to aid in separation thereof. The release liner can extend at least partially across the second surface of the inner layer.

A method of treatment includes the steps of:

(a) disposing a therapeutic drape over a wound, the therapeutic drape having an outer transparent layer and an inner layer for application adjacent a wound which includes a semi-permeable transparent adhesive cover removably connected to the outer transparent layer; and

(b) marking the outer layer about the wound.

The outer layer can include a printed grid thereon. The therapeutic drape can include a release liner sheet removably connected to the second surface of the inner layer and wherein the release liner is removed prior to performing step (a).

The method can include the step of (c): removing the outer layer from the inner layer; or forming at least one of an inlet port and a suction port in the therapeutic drape.

The method of treatment can include the step of (d): archiving the outer layer in a patient file; electronically archiving the outer layer in a patient data file; or forming at least one of an inlet port and a suction port in the inner layer; or employing the marked outer layer for purposes of resizing a new dressing for the wound.

### BRIEF DESCRIPTION OF THE DRAWINGS

FIG. 1 is a schematic illustrating the device of the invention.

FIG. 1A depicts a part of the invention.

FIG. 2 depicts a perspective view of a drape of the invention.

FIG. 3 depicts a bottom perspective view of the drape in FIG. 2.

FIG. 4 depicts an exploded view of the drape of the invention.

FIG. 5 depicts the drape of FIG. 2 disposed on a wound.

FIG. 6 depicts partial separation of components of the drape of FIG. 2

### DETAILED DESCRIPTION

As illustrated in the drawings, a disposable therapeutic device of the instant invention is generally designated by the numeral 10. The disposable therapeutic device 10 can preferably include a housing 12 which provides an improved therapeutic device with multiple uses and portability. The housing 12 can preferably be formed in a waterproof manner to protect components therein. In this regard, housing 12 can have a watertight sealed access panel 13 through which components can be accessed.

The device 10 can include a processor 14, which can be a microcontroller having an embedded microprocessor, Random Access Memory (RAM) and Flash Memory (FM). FM can preferably contain the programming instructions for a control algorithm. FM can preferably be non-volatile and retains its programming when the power is terminated. RAM can be utilized by the control algorithm for storing variables such as pressure measurements, alarm counts and the like, which the control algorithm uses while generating and maintaining the vacuum.

A membrane keypad and a light emitting diode LED or liquid crystal display (LCD) 16 can be electrically associated with processor 14 through a communication link, such as a cable. Keypad switches provide power control and are used to preset the desired pressure/vacuum levels. Light emitting diodes 17, 19 can be provided to indicate alarm conditions associated with canister fluid level, leaks of pressure in the dressing and canister, and power remaining in the power source.

Microcontroller 14 is electrically associated with, and controls the operation of, a first vacuum pump 18 and an optional second vacuum pump 20 through electrical connections. First vacuum pump 18 and optional second vacuum pump 20 can be one of many types including, for example, the pumps sold under the trademarks Hargraves® and Thomas®. Vacuum pumps 18 and 20 can use, for example, a reciprocating diaphragm or piston to create vacuum and can be typically powered by a direct current (DC) motor that can also optionally use a brushless commutator for increased reliability and longevity. Vacuum pumps 18 and 20 can be pneumatically associated with a disposable exudate collection canister 22 through a single-lumen tube 24.

In one embodiment, canister 22 has a volume which does not exceed 1000 ml. This can prevent accidental exsanguination of a patient in the event hemostasis has not yet been achieved at the wound site. Canister 22 can be of a custom design or one available off-the-shelf and sold under the trademark DeRoyal®.

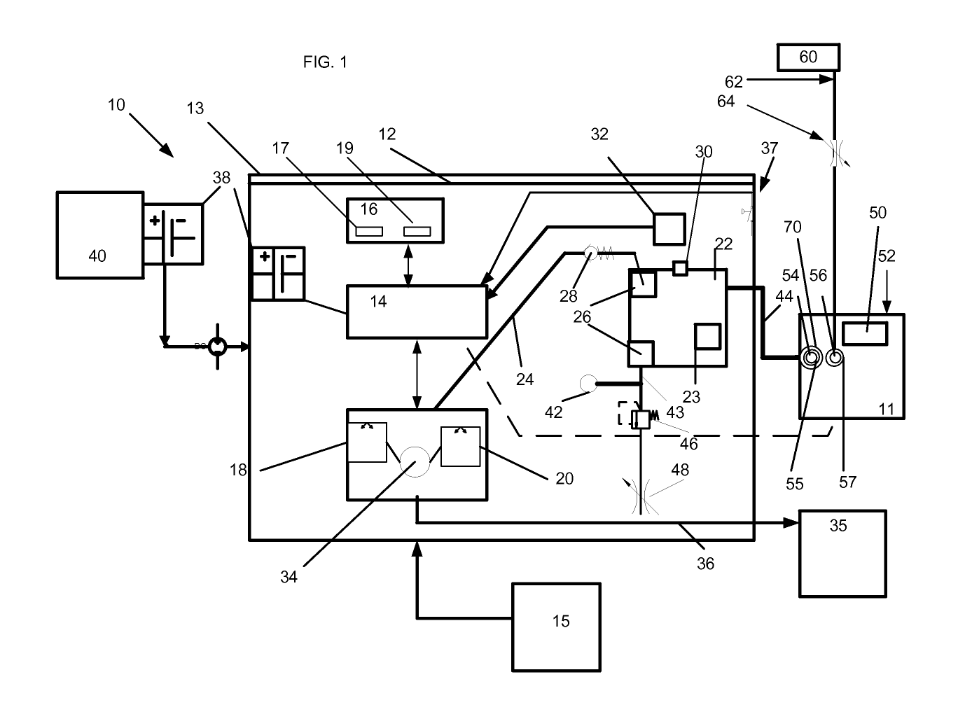

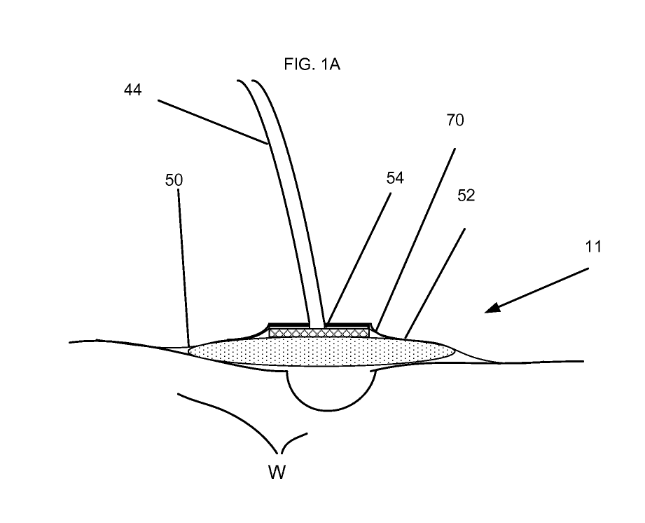

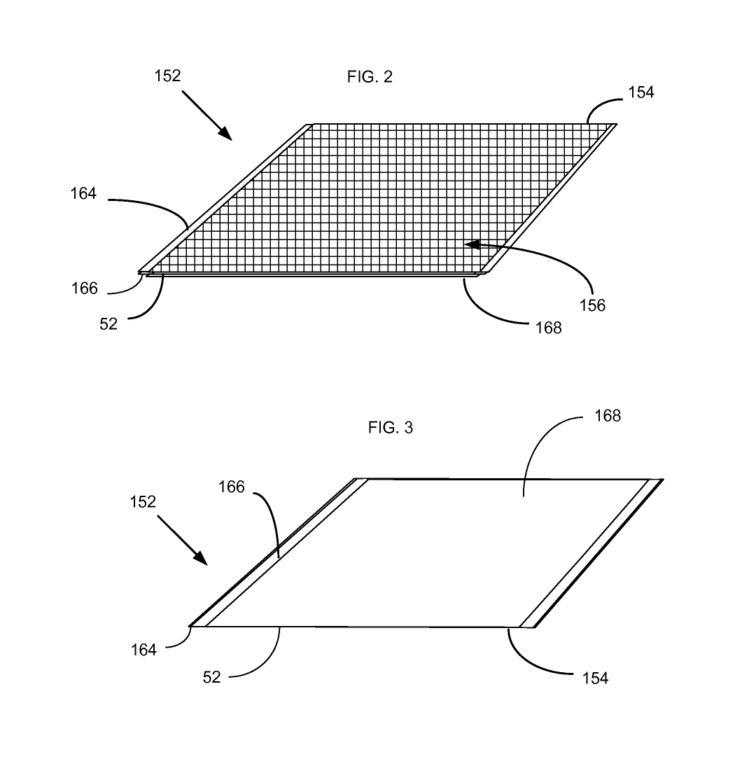

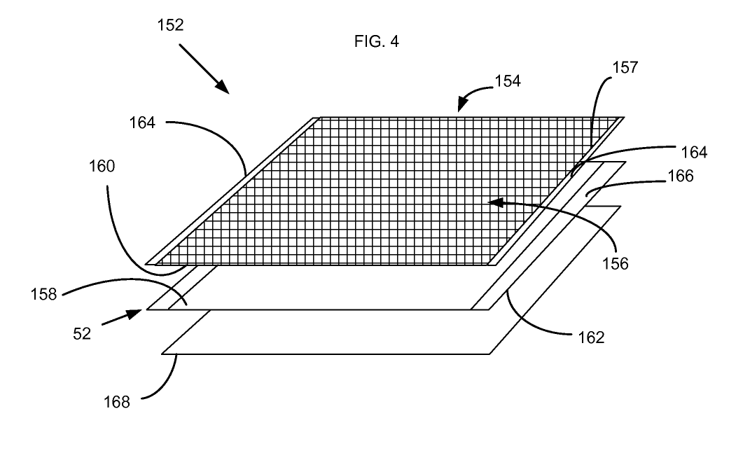

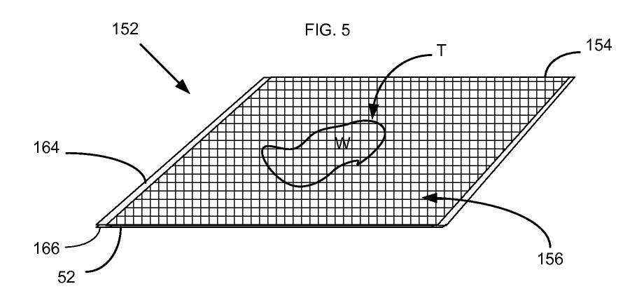

Referring now again to FIG. 1, a fluid barrier 26, which can be a back flow valve or filter, is associated with canister 22 and is configured to prevent fluids collected in canister 22 from escaping into tubing 24 and fouling the vacuum return path. Barrier 26 can be of a mechanical float design or may have one or more membranes of hydrophobic material such as those available under the trademark GoreTexTM. Barrier 26 can also be fabricated from a porous polymer such as that which is available under the trademark MicroPore™. A secondary barrier 28 using a hydrophobic membrane or valve is inserted in-line with pneumatic tubing 24 to prevent fluid ingress into the system in the event barrier 26 fails to operate as intended. Pneumatic tubing 24 can connect to first vacuum pump 18 and optional second vacuum pump 20 through “T” connectors.

An identification member 30, such as radio frequency identification (RFID) tag, can be physically associated with the canister 22 and an RFID sensor 32 operably associated with the microcontroller 14 such that the microcontroller 14 can restrict use of the device 10 to a predetermined canister 22. Thus, if a canister 22 does not have a predetermined RFID chip, the device 10 will not operate. Another embodiment envisions software resident on microcontroller 14 which restricts the use of the device 10 to a predetermined time period such as 90 days for example. In this way, the patient using the device 10 may use the device 10 for a prescribed time period and then the device 10 automatically times out per a particular therapeutic plan for that patient. This also enables a reminder of the time and date for the next dressing change or physician appointment. It is also contemplated that the microcontroller 14 be operably provided with a remote control 15 and communication link, such as a transceiver, wherein the device 10 can be shut down remotely when a particular therapeutic plan for that patient has ended. Likewise, remote control 15 can be utilized to provide additional time after the therapeutic device times out.

Vacuum-pressure sensor 34 is pneumatically associated with first vacuum pump 18 and optional vacuum pump 20 and electrically associated with microcontroller 14. Pressure sensor 34 provides a vacuum-pressure signal to the microprocessor enabling a control algorithm to monitor vacuum pressure at the outlet of the vacuum pumps 18 and 20.

An acoustic muffler can be provided and pneumatically associated with the exhaust ports of vacuum pumps 18 and 20 and configured to reduce exhaust noise produced by the pumps during operation. In normal operation of device 10, first vacuum pump 18 can be used to generate the initial or “draw-down” vacuum while optional second vacuum pump 20 can be used to maintain a desired vacuum within the system compensating for any leaks or pressure fluctuations. Vacuum pump 20 can be smaller and quieter than vacuum pump 18 providing a means to maintain desired pressure without disturbing the patient. It is contemplated by the instant invention that pumps 18 and 20 can also be employed to create a positive pressure for purposes of applying pressure to an inflatable member 35, such as a cuff or pressure bandage, through tubing 36. A switch 37 can be operatively disposed on housing 12 in operable connection with microcontroller 14 to enable selection of positive and negative pressure from pumps 18/20.

One or more battery (ies) 38 can preferably be provided to permit portable operation of the device 10. Battery 38 can be Lithium Ion (LiIon), Nickel-Metal-Hydride (NiMH), Nickel-Cadmium, (NiCd) or their equivalent, and can be electrically associated with microcontroller 14 through electrical connections. Battery 38 can be of a rechargeable type which is preferably removably disposed in connection with the housing 12 and can be replaced with a secondary battery 38 when needed. A recharger 40 is provided to keep one battery 38 charged at all times. Additionally, it is contemplated that the device 10 can be equipped to be powered or charged by recharger 40 or by circuits related with microcontroller 14 if such source of power is available. When an external source of power is not available and the device 10 is to operate in a portable mode, battery 38 supplies power to the device 10. The battery 38 can be rechargeable or reproces sable and can preferably be removably stored in a waterproof manner within housing 12 which also likewise contains the pumps 18, 20 and microcontroller 14.

A second pressure sensor 42 is pneumatically associated with canister 22 through a sensor port 43. Pressure sensor 42 can be electrically associated with microcontroller 14 and provides a vacuum-pressure signal to microprocessor enabling control algorithm to monitor vacuum pressure inside canister 22 and dressing 11. A “T” connector can be connected to port 43, to pressure sensor 42 and a vacuum-pressure relief solenoid 46 configured to relieve pressure in the canister 22 and dressing 11 in the event of an alarm condition, or if power is turned off. Solenoid 46, can be, for example, one available under the trademark Parker Hannifin® or Pneutronics®; Solenoid 46 is electrically associated with, and controlled by, microprocessor of microcontroller 14. Solenoid 46 can be configured to vent vacuum pressure to atmosphere when an electrical coil associated therewith is de-energized as would be the case if the power is turned off. An orifice restrictor 48 may optionally be provided in-line with solenoid 46 and pneumatic tube 44 to regulate the rate at which vacuum is relieved to atmospheric pressure when solenoid 46 is de-energized. Orifice restrictor 48 is, for example, available under the trademark AirLogic®.

A wound dressing 11 can preferably include a sterile porous substrate 50, which can be a polyurethane foam, polyvinyl alcohol foam, gauze, felt or other suitable material, semi-permeable transparent adhesive cover 52 which can be a plastic sheet of polyurethane material such as that sold under the trademark DeRoyal® or Avery Denison®. In this regard, a preferred embodiment of the invention provides a drape 152 which includes semi-permeable transparent adhesive cover 52.

The drape 152 can also preferably include an outer transparent layer 154, made of a plastic sheet of polyethylene for example, which preferably includes in one embodiment a printed grid 156 across an outer surface 157 thereof. The grid 156 can be symmetrically incremental and can optionally include numerical markings, although the grid 156 should preferably be marked in a manner to permit easy measurement of wound W. The drape 152 includes the transparent semi-permeable adhesive cover sheet 52 which forms inner layer having a first surface 158 which can be electrostatically bonded to inner surface 160 of the outer layer 154 and a second surface 162 which can be coated with an adhesive material, such as a pressure sensitive adhesive.

Opposing lateral edges 164 can be provided on layer 154 and opposing lateral edges 166 can similarly be provided on layer 154 wherein edges 164 and 166 can preferably be constructed in a non-stick manner. The lateral edges 164 and 166 can include a silicone or wax coat to aid in this regard.

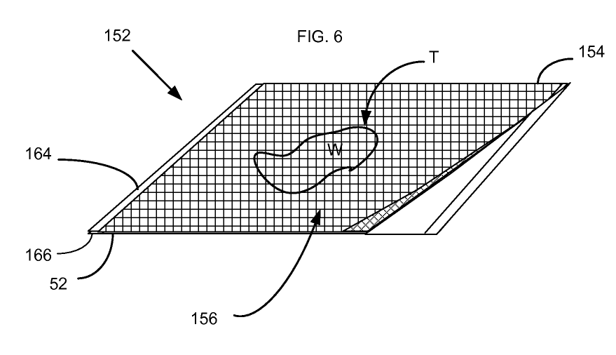

To the adhesive coated second surface 162 of layer 52 can be connected a release liner sheet of silicone treated paper-based substrate 168. The substrate 168 serves as a peel away backing which upon removal, as seen in FIG. 6, permits the adhesive coated second surface 162 of inner layer 52 to be applied over the wound site W. At this point a marker trace T can be made about the wound W onto the outer surface 157 of outer layer 154 of the drape 152. As seen in FIG. 7, the outer layer 154 can be peeled off away from the inner layer 52 and when removed, the layer 154 can be placed into a patients file to serve as a documenting aid for wound size estimation, dressing size estimations, and records purposes. Thus, the outer layer 154 can be archived in a patient file via placing into the file the original and/or scanning into an electronic file the outer layer 154.

The semi-permeable adhesive cover 52 can then be formed to include an inlet port 56 and a suction port 54. Substrate 50 is configured to distribute vacuum pressure evenly throughout the entire wound bed and has mechanical properties suitable for promoting the formation of granular tissue and approximating the wound margins.

In addition, when vacuum is applied to dressing 11, substrate 50 creates micro-and macro-strain at the cellular level of the wound stimulating the production of various growth factors and other cytokines, and promoting cell proliferation. Dressing 11 is fluidically associated with canister 22 through single-lumen tube 44. The vacuum pressure in a cavity formed by substrate 50 of dressing 11 is largely the same as the vacuum pressure inside canister 22 minus the weight of any standing fluid inside tubing 44.

A fluid vessel 60, which can be a standard IV bag, contains medicinal fluids such as aqueous topical antibiotics, analgesics, physiologic bleaches, or isotonic saline. Fluid vessel 60 is removably connected to dressing 11 though port 56 and single-lumen tube 62.

An optional flow control device 64 can be placed in-line with tubing 62 to permit accurate regulation of the fluid flow from vessel 60 to dressing 11. In normal operation, continuous wound site irrigation is provided as treatment fluids move from vessel 60 through dressing 11 and into collection canister 22. This continuous irrigation keeps the wound clean and helps to manage infection. In addition, effluent produced at the wound site and collected by substrate 50 will be removed to canister 22 when the system is under vacuum.

The device 10 is particularly well suited for providing therapeutic wound irrigation and vacuum drainage and provides for a self-contained plastic housing configured to be worn around the waist or carried in a pouch over the shoulder for patients who are ambulatory, and hung from the footboard or headboard of a bed for patients who are non-ambulatory. Membrane keypad and display 16 is provided to enable the adjustment of therapeutic parameters and to turn the unit on and off.

Depressing the power button on membrane switch 16 will turn the power to device 10 on/off. While it is contemplated that the membrane switch 16 be equipped with keys to adjust therapeutic pressure up and down, the microcontroller 14 can preferably be equipped to control the pressure in accordance with sensed pressure and condition to maintain pressure in an operable range between −70 mmHg and −150 mmHg with a working range of between 0 and −500 mmHg, for example. Although these pressure settings are provided by way of example, they are not intended to be limiting because other pressures can be utilized for wound-type specific applications. The membrane 16 can also be equipped with LED 17 to indicate a leak alarm and/or LED 19 indicates a full-canister alarm. When either alarm condition is detected, these LEDs will light in conjunction with an audible chime which is also included in the device 10.

Housing 12 can incorporate a compartment configured in such a way as to receive and store a standard IV bag 60 or can be externally coupled to thereto. IV bag 60 may contain an aqueous topical wound treatment fluid that is utilized by the device 60 to provide continuous irrigation. A belt clip can provided for attaching to a patient's belt and an optional waist strap or shoulder strap is provided for patients who do not or cannot wear belts.

Canister 22 is provided for exudate collection and can preferably be configured as currently known in the field with a vacuum-sealing means and associated fluid barrier 26, vacuum sensor port 43 and associated protective hydrophobic filter, contact-clear translucent body, clear graduated measurement window, locking means and tubing connection means. Collection canister 22 typically has a volume less than 1000 ml to prevent accidental exsanguination of a patient if hemostasis is not achieved in the wound. Fluid barriers 26 can be, for example, those sold under the trademark MicroPore® or GoreTex® and ensure the contents of canister 22 do not inadvertently ingress into pumps 18, 20 of housing 12 and subsequently cause contamination of thereof.

Pressure sensor 42 enables microcontroller 14 to measure the pressure within the canister 22 as a proxy for the therapeutic vacuum pressure under the dressing 11.

Optionally, tubing 62 can be multilumen tubing providing one conduit for the irrigation fluid to travel to dressing 11 and another conduit for the vacuum drainage. Thus, IV bag 60, tubing 62, dressing 11 and canister 22 provide a closed fluid pathway. In this embodiment, canister 22 would be single-use disposable and may be filled with a solidifying agent 23 to enable the contents to solidify prior to disposal. Solidifying agents are available, for example, under the trademark DeRoyal® and Isolyzer®. The solidifying agents prevent fluid from sloshing around inside the canister particularly when the patient is mobile, such as would be the case if the patient were travelling in a motor vehicle. In addition, solidifying agents are available with antimicrobials that can destroy pathogens and help prevent aerosolization of bacteria.

At the termination of optional multilumen tubing 62, there can be provided a self-adhesive dressing connector 57 for attaching tubing 62 to semi-permeable transparent adhesive cover 52 to provide a substantially air-tight seal. Dressing connector 11 can have an annular pressure-sensitive adhesive ring with a release liner that is removed prior to application. Port 56 can be formed as a hole cut in a semi-permeable transparent adhesive cover 52 and dressing connector 57 would be positioned in alignment with said hole. This enables irrigation fluid to both enter and leave the dressing through a single port. In an alternative embodiment, tube 62 can bifurcate at the terminus and connect to two dressing connectors 57 which allow the irrigation port to be physically separated from the vacuum drainage port thus forcing irrigation fluid to flow though the entire length of the dressing if it is so desired. Similarly, port 54 and connector 55 can be provided to connect optional multilumen tubing 44 to dressing 11. In this arrangement, the second lumen may be used to directly measure the pressure in dressing 11.

Fluid vessel 60 can be of the type which includes a self-sealing needle port situated on the superior aspect of the vessel 60 and a regulated drip port situated on the inferior aspect of the vessel. The needle port permits the introduction of a hypodermic needle for the administration of aqueous topical wound treatment fluids. These aqueous topical fluids can include a topical anesthetic such as Lidocaine, antibiotics such as Bacitracin or Sulfamide-Acetate; physiologic bleach such as Chlorpactin or Dakins solution; and antiseptics such as Lavasept or Octenisept. Regulated drip port permits fluid within vessel 60 to egress slowly and continuously into porous substrate 50 whereupon the therapeutic benefits can be imparted to the wound site. Single-lumen drainage tube 44 provides enough vacuum to keep the dressing 11 at sub-atmospheric pressure and to remove fluids, which include the irrigation fluid and wound exudates. With this modification, the need for an external fluid vessel and associated tubing and connectors can be eliminated making the dressing more user friendly for patient and clinician alike.

In typical clinical use of this alternate embodiment, dressing 11 is applied to the wound site by first cutting porous substrate 50 to fit the margins of the wound. Next, a semi-permeable transparent adhesive cover 52 is attached and sealed over the dressing and periwound. A hole approximately 3/8 ″ diameter can be made in a semi-permeable transparent adhesive cover 52 central to porous substrate 50. Fluid vessel 60 is attached by adhesive annular ring 57 with port 56 aligned with the hole previously cut in a semi-permeable transparent adhesive cover 52. Once the fluid vessel 60 is hermitically sealed to the semi-permeable transparent adhesive cover 52, a properly prepared hypodermic needle is inserted in self-sealing needle port and fluid vessel 60 subsequently filled with the desired aqueous topical wound treatment solution.

For the majority of applications, the technique for providing therapeutic wound irrigation and vacuum drainage is illustrated. The single lumen drainage tube 44 is provided for the application of vacuum and removal of fluids from the wound site. Fluid vessel 60 can be situated outside and superior to semi-permeable substrate 50. An annular adhesive ring 57 is provided on port 56 for attachment of single-lumen irrigation tubing 62 to a semi-permeable transparent adhesive cover 52. Similarly, a needle port permits the introduction of a hypodermic needle for the administration of aqueous topical wound treatment fluids as described above, for example, a caregiver may want to add a topical antibiotic to a bag of isotonic saline. Adjustable optional flow control device 64 permits fluid within vessel 60 to egress slowly and continuously into porous substrate 50 through hole 56 in a semi-permeable transparent adhesive cover 52 whereupon the therapeutic benefits can be imparted to the wound site. Single-lumen drainage tube 44 provides enough vacuum to keep the dressing 11 at sub-atmospheric pressure and to remove fluids which include the irrigation fluid and wound exudates.

Because of the potential chemical interactions between the various materials used in the construction of dressing 11, attention must be paid to the types of aqueous topical wound fluids used to ensure compatibility.

The above described embodiments are set forth by way of example and are not limiting. It will be readily apparent that obvious modifications, derivations and variations can be made to the embodiments. For example, the vacuum pumps described having either a diaphragm or piston-type could also be one of a syringe based system, bellows, or even an oscillating linear pump. Accordingly, the claims appended hereto should be read in their full scope including any such modifications, derivations and variations.

1. A therapeutic drape, which includes:
an outer transparent layer which has a printed grid across an outer surface thereof; and
an inner layer for application adjacent a wound which includes a semi-permeable transparent adhesive cover removably connected to said outer transparent layer.

2. The therapeutic drape of claim 1, wherein said semi-permeable transparent adhesive cover has a first surface connected to said outer layer and a second surface having a pressure sensitive adhesive material thereon.

3. The therapeutic drape of claim 2, which includes a release liner sheet removably connected to said second surface of said inner layer.

4. The therapeutic drape of claim 1, wherein at least one of said outer layer and said inner layer includes opposing lateral edges configured in a non-stick manner to aid in separation of said layers.

5. The therapeutic drape of claim 1, wherein said outer layer and said inner layer are one of removably adhesively and electrostatically connected.

6. The therapeutic drape of claim 3, wherein said outer layer, said inner layer and said release liner are one of removably adhesively and electrostatically interconnected.

7. The therapeutic drape of claim 1, wherein said outer layer and said inner layer are made of plastic material.

8. The therapeutic drape of claim 3, wherein said outer layer and said inner layer are made of plastic material.

9. The therapeutic drape of claim 1, wherein said grid is further characterized to be symmetrical.

10. A method of treatment, which includes the steps of:
(a) disposing a therapeutic drape over a wound, said therapeutic drape having an outer transparent layer and an inner layer for application adjacent a wound which includes a semi-permeable transparent adhesive cover removably connected to said outer transparent layer; and
(b) marking said outer layer about said wound.

11. The method of treatment of claim 10, which includes the step of (c) removing said outer layer from said inner layer.

12. The method of treatment of claim 10, wherein said outer layer includes a printed grid thereon.

13. The method of treatment of claim 10, wherein said therapeutic drape includes a release liner sheet removably connected to said second surface of said inner layer and wherein said release liner is removed prior to performing step (a).

14. The method of treatment of claim 11, which includes the step of (d) archiving said outer layer in a patient file.

15. The method of treatment of claim 11, which includes the step of (d) electronically archiving said outer layer in a patient data file.

16. The method of treatment of claim 11, which includes the step of (d) employing said marked outer layer for purposes of resizing a new dressing for the wound.

17. The method of treatment of claim 10, which includes the step of (c) forming at least one of an inlet port and a suction port in said therapeutic drape.

18. The method of treatment of claim 11, which includes the step of (d) forming at least one of an inlet port and a suction port in said inner layer.

19. The method of treatment of claim 13, which includes the step of (c) forming at least one of an inlet port and a suction port in said therapeutic drape.

20. The method of treatment of claim 13, which includes the step of (c) removing said outer layer from said inner layer and (d) forming at least one of an inlet port and a suction port in said inner layer.
